(19) United States
(12) Patent Application Publication
(10) Pub. No.: US 2005/0257327 A1
(43) Pub. Date: Nov. 24, 2005

(54) Method for dyeing with sulfur and sulfur vat dyes

(75) Inventors: Thomas Bechtold, Dornbirn (AT); Wolfgang Schrott, Ludwigshafen (DE); Thorsten Huls, Hofheim (DE); Marc-Steffen Muche, Frankfurt (DE); Bertram Wendt, Konigstein (DE)

Correspondence Address: CONNOLLY BOVE LODGE & HUTZ, LLP, P O BOX 2207, WILMINGTON, DE, 19899, US

(73) Assignee: DyStar Textilfarben GmbH & Co. Deutschland KG, Frankfurt am Main (DE)

(21) Appl. No.: 10/521,917

(22) PCT Filed: Jul. 23, 2003
(86) PCT No.: PCT/EP03/08050

§ 371 Date: Jan. 20, 2005

(30) Foreign Application Priority Data
Jul. 31, 2002 (DE) 102 34 825.1

(51) Int. Cl. A61K 7/13

(52) U.S. Cl. 8/405

(57) ABSTRACT

The present invention relates to a process for dyeing fiber materials with sulfur dyes by regenerating the dyebath redox potential, which comprises, during the dyeing process, the dyeing liquor being circulated between the dyeing apparatus and an attached electrolytic cell and the sulfur dye which has been unwantedly oxidized in the dyebath being cathodically reduced in the electrolytic cell.

The present invention relates to a process for dyeing fiber materials with sulfur dyes and sulfur vat dyes.

The group of sulfur dyes and sulfur vat dyes (hereinafter referred to as just sulfur dyes) groups dyes of the same method of manufacture and the same method of dyeing together. Sulfur dyes are produced by reaction of suitable organic substances with sulfur, alkali metal sulfides or alkali metal polysulfides. The products formed contain recurring organic structural elements which are joined together by disulfide groups. The chemical constitution is in most cases not certain. For dyeing, sulfur dyes are reduced by various methods which reductively cleave a portion of the disulfide bridges (see equation 1). The products formed have lower molar masses, are soluble in aqueous alkaline solution and can be used for dyeing since they also have an affinity for fibers, for example cellulosic fibers.

A more or less complete back-oxidation of the dye takes place in the presence of atmospheric oxygen, in accordance with equation 2.
R—S—S—R+2e⁻⇄R—S⁻+R—S⁻ (1)
R—S⁻+R—S⁻+1/2O₂+H₂O⇄R—S—S—R+2OH⁻ (2)

Since, in the dyeing operation, the dyebath containing the reduced dye has to be protected against unwanted oxidation of the dye by air, either reducing chemicals are introduced into the dyebath or a more far-reaching cathodic dye reduction is carried out during dye manufacture or the preparation of the dyeing liquor (see WO 99/11716). The procedure of WO 99/11716 makes it possible to dispense with the continued use of reducing agents in the production of reduced dyes and their use in continuous dyeing processes provided the dye concentration employed is sufficiently high, for example 50 g/l of solid sulfur dye, so that the reduction equivalents introduced into the dye liquor together with the reduced dye are able to compensate the disruptive influence of air oxidation. Such an approach is particularly useful for the production of relatively concentrated products or dye liquors which are only briefly exposed to the oxidative action of atmospheric oxygen during continuous dyeing. A dye box holding 25 l of dye liquor is turned over within less than 3 min at a customary fabric speed of 60 m/min, a linear meter weight of 200 g/m² and a wet pickup of 80%.

There is no prior art technique for applying sulfur dyes in exhaust dyeing, for example on circulating liquor/stationary goods machines, jet dyeing machines, etc, which by analogy also includes the continuous warp yarn dyeing ranges for denim manufacture. The long dyeing time is responsible for long residence times of the dye in the dyebath, which is exposed to continual oxidative action of atmospheric oxygen during this period. In addition, dye concentrations used in exhaust dyeing are relatively low at the commencement of dyeing and decrease further during the dyeing process by reason of bath exhaustion. The instability of the dyebath to undesirable atmospheric oxidation accordingly increases more and more as the dyeing progresses.

By way of illustration, a typical example will now be calculated for a dark exhaust dyeing:

To dye 1 kg of fiber material to a depth of 5% (reckoned as solid sulfur dye) at a liquor ratio of 10:1, a total of 50 g of dye will be included per 10 l of dyebath, so that the initial concentration of dye in the dyebath will be 5 g/l. Assuming a bath exhaustion of 70% for the dyeing operation, the concentration of dye will have decreased to 1.5 g/l of dye by the end of the dyeing process. In dyeing processes which are known from the prior art, exhaust bath stabilization to oxidative influences can therefore only be achieved through the addition of appropriate amounts of reducing chemicals such as glucose or hydroxyacetone.

If these additives are not used, the sulfur dye will undergo uncontrolled back-oxidation during the dyeing operation. Observable consequences are poor reproducibility of the depth of shade, unlevelness and poor crockfastnesses.

Warp dyeing ranges customarily contain relatively high use concentrations of dye (50 g/l of solid dye) and relatively high liquor volumes, so that bath stability to atmospheric oxygen appears to be higher. But these dyeing techniques require the baths to have a very long use time, since it is customary to enter the dyebath with wet fabric and so only small amounts of the dyeing liquor are carried out of the dyebaths. A bath volume of 4 000 l and a production of 15000 kg of warp yarn per day and assuming a squeeze-off effect of 70% at prewetting and of 95% at dyeing results in a liquor consumption of 15 000×0.25=3 750 l per day, so that the average residence time for the dyeing liquor in the dyeing range turns out to be one day. If no reducing agents were used, the phenomenon of ending (that is, hue changes within a dyeing lot of 20 000 m lengths for example) would be inevitable.

There are also literature proposals for using indirect cathodic reducing methods. See for example Textilveredlung 32 (1997) 204-209, Journal of Applied Electrochemistry 28 (1998) 1243-1250, Recent Res. Devel. in Electrochem. 1 (1998) 245-264 and WO 90/15182. In these processes, a regenerable redox system performs the function of the soluble reducing agent, so that the required bath stability is ensured. Examples of such systems are anthraquinonoids, iron complexes with amines or hydroxy carboxylic acids. But with these processes too it is not possible to dispense with the use of chemicals.

The present invention, then, rests on the surprising discovery that sulfur dyes can perform the function of a mediator in exhaust dyeings too, and adequate bath stability can be achieved, when ongoing regeneration of the reducing state can be achieved. This is achieved according to the present invention when adequate circulation of the dyebath through a suitably attached electrolytic cell is made possible during the dyeing process.

The present invention accordingly provides a process for dyeing fiber materials with sulfur dyes by regenerating the dyebath redox potential, which comprises, during the dyeing process, the dyeing liquor being circulated between the dyeing apparatus and an attached electrolytic cell and the sulfur dye which has been unwantedly oxidized in the dyebath being cathodically reduced in the electrolytic cell.

The process of the present invention can be executed for example as an exhaust process, or else as a continuous process. Useful dyeing apparatus includes accordingly, for the exhaust process, circulating liquor/stationary goods machines, for example yarn dyeing machines, reel becks, beam dyeing machines and jet or overflow dyeing machines. For the continuous process, in contrast, the dyeing ranges which are customary for this process are used.

The dyebath has to be circulated between the dyeing apparatus and the electrolytic cell in accordance with the dye concentration and the oxidative burden. When the oxidative burden is high and the dye concentration low, the liquor has to be circulated at a higher volume flow rate than when the dye concentration is high and the oxygen burden low.

The cathodically reduced dye passes from the electrolytic cell to the dyeing apparatus and the partially oxidized dyebath flows from the dyeing apparatus to the electrolytic cell. The requisite liquor exchange in I/min between the electrolytic cell and the dyeing apparatus depends on plural general conditions. These include for example dye concentration, desired degree of reduction in the dyeing apparatus, maximum degree of reduction achievable for a sulfur dye by cathodic reduction, the minimum degree of sulfur dye reduction required for dyeing, the current density which can be used with the given cell, and also the oxygen input into the dyeing apparatus (oxidative burden).

When concentrations of sulfur dye are high, as is usually the case in warp yarn dyeing operations, it is also possible to contemplate a batchwise regeneration of the sulfur dye and hence an intermittent bath circulation.

A person of ordinary skill in the art is readily able to calculate the requisite mass transfer between cell and dyeing apparatus given knowledge of the present invention and of the essential general conditions mentioned.

If, for example, a current strength of 10A per kg of fiber is assumed as being necessary to compensate the oxygen input and if the amount of dye available in the dyebath circulation is put at 0.01 mol/l, then a dyebath circulation of 5 l/min is needed in order that the conversion achieved in the cell may not increase to above 10% of the existing dye concentration. A circulation rate of 10 l/min kg will change the dye solution in the reduced state by 5% only.

Depending on general conditions, the liquor exchange per kg of fiber will vary between 0.5 l/min kg and 100 l/min kg, preferably between 1 and 50 l/min kg and most preferably between 5 and 30 l/min kg.

The dye concentration in the dyebath in the process of the present invention is preferably in the range from 0.5 to 100 g/l of pure dye and more preferably in the range from 5 to 50 g/l of pure dye.

The process of the present invention is advantageously carried out at temperatures from 20 to 135° C. and more preferably at temperatures from 60 to 95° C.

In a preferred embodiment of the process according to the present invention, the dyeing operation is influenced by open loop control of the redox potential. This is accomplished by adjusting the cell current, making it possible to change or closed loop control the redox potential in the dyebath within certain potential limits. The adjustable potential range is determined by the sulfur dye used, its concentration and also by pH and dyeing temperature.

The cell current is defined in particular by the oxygen input and varies between 0.5 and 50 A/kg and preferably between 1 and 10 A/kg for customary dyeing apparatus. By employing suitable measures, such as a protective gas atmosphere of nitrogen for example, the values can be lowered.

The dyebath pH is for example between 9 and 14 and preferably between 11 and 13.

The redox potential in the dyebath is defined by the dye and the desired dyeing outcome and is between −300 mV and −900 mV and preferably between −400 mV and −700 mV.

The dyeing apparatus has attached to it an electrolytic cell with liquor circulation. The electrolytic cell used can be any electrolytic cell available from cell manufacturers or on the market. Normal or else multicathode cells can be used. To avoid anodic back-oxidation of the sulfur dye, however, the electrolytic cell is preferably constructed as a divided cell, and in turn it is particularly preferable to use a membrane electrolytic cell. Most preferably, a cation exchange membrane is used as separator.

The conducting electrolyte used is preferably selected from alkaline solutions, preferably alkaline solutions of alkali metal salts, especially of sodium hydroxide, potassium hydroxide, sodium carbonate, sodium chloride or sodium sulfate. Particular preference is given to using the alkali added to the dyebath, advantageously aqueous sodium hydroxide solution, aqueous potassium hydroxide solution or sodium carbonate. Similarly, salts added during dyeing, preferably sodium chloride or sodium sulfate, can improve conductivity as electrolytes.

In a further preferred embodiment of the process according to the present invention, this process is carried out under an inert atmosphere. To this end, the dyebath in the dyeing apparatus is blanketed with nitrogen or a noble gas and more preferably argon.

Since the basic oxidative burden is reduced by reducing the partial pressure of atmospheric oxygen, it is thus possible to dimension the requisite electrolytic cells with smaller cell currents and hence more economically.

The process of the present invention is unreservedly useful for all sulfur dyes. Not only oxidized dyes, as-synthesized filter cakes but also cathodically or chemically prereduced dyes and dye preparations can be used. Particular preference is given to sulfur dyes produced by cathodic reduction as are described for example in DE-A 1 906 083 or WO 99/11716.

The process according to the present invention can be used to dye all fiber materials which are sulfur dyeable in principle. These are in particular fiber materials composed of cellulose and polyamide and also of cellulose-polyester and cellulose-polyamide blends. Fiber materials preferably refers to textile fiber materials.

When dyeing with sulfur dyes, atmospheric oxygen introduced into the dyebath is reduced by the reduced sulfur dye present. In the process according to the present invention, the redox behavior of sulfur dyes, which is characterized by a plurality of reduction states (see for example Journal of Applied Electrochemistry 28 (1998) 1243-1250 and Recent Res. Devel. in Electrochem. 1 (1998) 245-264), is advantageously exploited by working with adequate cell circulation and cathodic supplementary reduction of the oxidized sulfur dye, so that stable bath states are realized.

In the process according to the present invention, the sulfur dye performs the function of the reducing agents or cathodically regenerable mediators hitherto considered indispensable in exhaust dyeing. It is therefore possible to dispense with the use of chemicals which create costs in procurement and wastewater treatment, and an advantageous ecological overall balance is achieved.

Unexpectedly, the low concentrations of sulfur dye used in exhaust processes are sufficient to carry out the process according to the present invention. It is very particularly advantageous to practice the process of the present invention when dyeing from a stationary bath, it merely being necessary to replenish the dyebath with the sulfur dye carried out with the fabric.

The use examples 1-5 which follow illustrate typical possibilities for the process according to the present invention. To achieve a clear demonstration of its effect, the sample dyeings were commenced with oxidized sulfur dye which is not directly suitable for dyeing and only able to go onto the material after cathodic reduction.

### USE EXAMPLE 1

### Exhaust Dyeing with Sulfur Black 1

The electrolytic cell used was a cell divided by a cation exchange membrane. Cathode: stainless steel cathodes, total cathode surface area 0.43 m², volume in total 2 l.

Anode: stainless steel plate 0.01 m² in area. Volume 0.3 l.

The anolyte used is 0.1 M NaOH.

Cell current: 0.9 A, cell voltage between 2.7 V and 4.1 V

The dyebath (total volume 2 l) is pumped at 150 ml/min through the cathode space, so that an ongoing regeneration of the dyebath takes place through exchange with the catholyte.

Dyebath/Catholyte Composition:

10 g/l of Cassulfon® Carbon CMR paste from DyStar Textilfarben GmbH & Co. Deutschland KG

0.6 g/l of wetting agent

3 g/l of NaOH

The dyebath contains a bleached cotton drawn-loop knit (sample 1) having a mass of 6.9 g. Liquor circulation and heating is provided by a magnetic stirrer. The catholyte temperature is brought to 70° C. During an electrolysis time of 197 min, the redox potential decreases from −259 mV (vs. Ag/AgCl, 3 M KCl reference) to −499 mV. The dyed sample 1 is removed, rinsed with water and oxidized with peroxide/acetic acid as per usual.

The dyebath is entered with a further sample (sample 2, mass 6.9 g) which is dyed for 30 min by continuing the electrolytic operation. The redox potential decreases to −545 mV. Sample 2 is removed after 30 min and finished as already described.

Dyebath pH is about 1 2.2

The depth of shade can be described by color locus measurement.

Results:

As the L values show, sample 2 is darker even though the dyeing time was shorter. This is attributable to the continued buildup of the redox potential in the dyebath. Despite the low dye concentration, this confirms successful dye reduction under the conditions of exhaust dyeing.

| | L | a | b |
|---|---|---|---|
| | (lightness) | (red-green) | (yellow-blue) |
| Sample 1 | 26.06 | −1.02 | −3.42 |
| Sample 2 | 22.07 | −1.34 | −2.66 |

### USE EXAMPLE 2

### Exhaust Dyeing with Sulfur Black 1

The electrolytic cell used was a cell divided by a cation exchange membrane. Cathode: stainless steel cathodes, total cathode surface area 0.43 m², volume in total 2 l.

Anode: stainless steel plate 0.01 m² in area. Volume 0.3 l.

The anolyte used is 0.1 M NaOH.

Cell current: 0.9 A, cell voltage between 3.0 V and 4.7 V

The dyebath (total volume 2 l) is pumped at 150 ml/min through the cathode space, so that an ongoing regeneration of the dyebath takes place through exchange with the catholyte.

Dyebath/catholyte composition:

10.5 g/l of Cassulfon® Carbon CMR paste from DyStar Textilfarben GmbH & Co. Deutschland KG

0 6 g/l of wetting agent

3 g/l of NaOH

The dyebath contains a bleached cotton drawn-loop knit (sample 3) having a mass of 6.8 g. Liquor circulation and heating is provided by a magnetic stirrer. The catholyte temperature is brought to 62-64° C. During an electrolysis time of 175 min, the redox potential decreases from −309 mV (vs. Ag/AgCl, 3 M KCl reference) to −440 mV. The dyed sample 3 is removed, rinsed with water and oxidized with peroxide/acetic acid as per usual.

The dyebath is entered with a further sample (sample 4, mass 7.0 g) which is dyed for 80 min by continuing the electrolytic operation. The redox potential decreases to −437-−431 mV. Sample 4 is removed after 80 min and finished as already described.

Dyebath pH is about 12.1-12.2.

The depth of shade can be described by color locus measurement.

Results:

As the L values show, sample 4 is darker even though the dyeing time was shorter. This is attributable to the continued buildup of the redox potential in the dyebath. Despite the low dye concentration, this confirms successful dye reduction under the conditions of exhaust dyeing.

| | L | a | b |
|---|---|---|---|
| | (lightness) | (red-green) | (yellow-blue) |
| Sample 3 | 38.00 | −1.05 | −3.71 |
| Sample 4 | 31.58 | 0.97 | −3.64 |

### USE EXAMPLE 3

### Dyeing on a Laboratory Denim Dyeing Range

Electrolytic cell:

The electrolytic cell used is a cell divided by a cation exchange membrane.

Cathode: stainless steel cathodes, total cathode surface area 1 m², total catholyte volume 10 l.

Anode: titanium electrode with mixed oxide coating, expanded metal having geometric surface area of 0.04 m². Volume 1.5 l.

The anolyte used is 1 M NaOH.

Cell current: 10 A, cell voltage between 3.0 V and 4.7 V.

A Looptex laboratory dyeing range for denim dyeings is coupled to the cell. After an electrolysis time of 17.5 h at 10 A (75 Ah) to reach the dyeing potential, a portion of the catholyte (4 l) is pumped from the cell into the dyeing range and samples 5 and 6 are dyed at respectively 50° C. (−491 mV) and 80° C. (−567 mV) (yarn strands 150 m in length, raw cotton yarn).

Dyeing program: prewetting (3 g/l of wetting agent), squeezing off, dipping in the sulfur vat, squeezing off, air oxidation, subsequent rinse in cold water.

After dyeings 5 and 6, the dyebath is pumped back into the cell and again reduced by cathodic reduction.

After a reduction time of 3.7 h at 10 A (3.7 Ah), again a portion of the cell contents is pumped into the dyeing range and samples 7 and 8 are dyed according to the previously described program at respectively 57° C./−538 mV and 83° C./−536 mV.

Total volume of dyebath: 12 l

Dyebath/catholyte composition:

80.25 g/l of Sulfur Black 1 filter cake (50% water content)

2.0 g/l of wetting agent

4 ml/l of 50% aqueous sodium hydroxide solution

By regenerating the bath contents it is thus possible to ensure maintenance of the reduced state.

Dyebath pH is about 12.5-12.7.

The depth of shade can be described by color locus measurement.

Results:

| | Dyebath | L | a | b |
|---|---|---|---|---|
| | potential mV | (lightness) | (red-green) | (yellow-blue) |
| Sample 5 | −491 | 25.54 | −1.40 | −4.24 |
| Sample 6 | −567 | 22.18 | −0.69 | −3.07 |
| Sample 7 | −538 | 22.35 | −0.85 | −3.39 |
| Sample 8 | −536 | 18.54 | −0.18 | −2.29 |

### USE EXAMPLE 4

### EC Dyeing of EC-Reduced Sulfur Black

A solution of 20 ml/l of Cassulfon® Carbon CMR from DyStar Textilfarben GmbH & Co. Deutschland KG (about. 30-40% solution of Leuco Sulfur Black 1) is electrolyzed at pH 12 and room temperature in apparatus as described in use example 1 in the presence of 20 g/l of Na₂SO₄ anhydrous. The anolyte used is again aqueous sodium hydroxide solution (40 g/l of NaOH). The solution of the reduced sulfur dye has a reducing agent equivalents content of 0.075 mol/l (determined by iodometric titration) at the start of electrolysis. The cathodic reduction is carried out at a current density of 0.26 mA/cm² in line with the low sulfur dye content of the catholyte. The electrolysis is ended at an analytically determined content of 0.125 mol/l. The solution then has a reducing agent equivalents content of 335 Ah based on 1 kg of solid sulfur dye. The thus prepared solution of the sulfur dye can be used directly for dyeing, for example as described in use example 1.

### USE EXAMPLE 5

### Exhaust Dyeing with Sulfur Black 1 on a Dyeing Jet Under Protective Gas (Nitrogen Atmosphere)

The electrolytic cell used is a cell divided by a cation exchange membrane. Cathode: three-dimensional stainless steel cathodes, visible cathode dimensions 60×55 cm, area 0.33 m², total volume of cathode space 100 l.

Anode: titanium electrode with platinum mixed oxide coating having 0.3 m² area. The anolyte used is 0.1 M NaOH.

Cell current: 85 A, cell voltage between 5.3 V and 5.7 V

The dyebath (total volume 230 l) is pumped through the cathode space, so that an ongoing regeneration of the dyebath or of the reduced dye takes place through exchange with the catholyte.

Dyebath/catholyte composition: 4.5 g/l of Cassulfon® Carbon CMR paste from DyStar Textilfarben GmbH & Co. Deutschland KG (=electrochemically prereduced dye)

1.0 g/l of wetting agent

7 g/l of 380 Bé caustic soda

The dyebath contains a prewashed bleached cotton drawn-loop knit having a mass of 8 kg. Liquor circulation and fabric agitation are provided by the pump associated with the jet. Heat is supplied by an indirect steam heating system. The dyeing was carried out under protective gas atmosphere (nitrogen) in order that air access may be minimized. To this end, a 10 i/min nitrogen stream is continuously passed into the apparatus.

Fabric speed is 50 m/min. Liquor circulation through the cell is 30 l/min. The catholyte temperature is brought to about 55° C., after which cell circulation is connected up and heating continues to 76° C. During an electrolysis time of about 80 min, the redox potential is between −630 mV and −720 mV when measured in the cell and between −460 mV and −432 mV when measured in the dyeing jet (vs. Ag/AgCl, 3 M KCl reference).

Dyebath pH is about 12.1-12.2.

After overflow rinsing, the black dyeing is finished in conventional manner, for example by oxidation with hydrogen peroxide/acetic acid, rinsing and buffering.

1. A process for dyeing fiber materials with sulfur dyes by regenerating the dyebath redox potential, which comprises, during the dyeing process, the dyeing liquor being circulated between the dyeing apparatus and an attached electrolytic cell and the sulfur dye which has been unwantedly oxidized in the dyebath being cathodically reduced in the electrolytic cell.

2. A process as claimed in claim 1, wherein the dyebath redox potential is closed loop controlled by the cell current.

3. A process as claimed in claim 1, wherein the electrolytic cell used is a divided electrolytic cell and more advantageously a membrane electrolytic cell.

4. A process as claimed in claim 1, wherein the conducting electrolyte used is selected from alkaline solutions.

5. A process as claimed in claim 1, wherein the dye concentration in the dyebath is in the range from 0.5 to 100 g/l of pure dye.

6. A process as claimed in claim 1, conducted at a temperature in the range from 20 to 135° C.

7. A process as claimed in claim 1, conducted under an inert atmosphere.

8. A process as claimed in claim 1, wherein the fiber materials used are fiber materials composed of cellulose or polyamide or cellulose-polyester or cellulose-polyamide blends.

9. A process as claimed in claim 2, wherein the electrolytic cell used is a divided electrolytic cell and more advantageously a membrane electrolytic cell.

10. A process as claimed in claim 9, wherein the conducting electrolyte used is alkaline solutions of alkali metal salts.

11. A process as claimed in claim 10, wherein the conducting electrolyte used is sodium hydroxide, potassium hydroxide, sodium carbonate, sodium chloride or sodium sulfate.

12. A process as claimed in claim 11, wherein the dye concentration in the dyebath is in the range from 5 to 50 g/l of pure dye.

13. A process as claimed in claim 12, conducted at a temperature in the range from 60 to 95° C.

14. A process as claimed in claim 13, conducted under an inert atmosphere.

15. A process as claimed in claim 14, wherein the fiber materials used are fiber materials composed of cellulose or polyamide or cellulose-polyester or cellulose-polyamide blends.
